(19) United States
(12) Patent Application Publication
(10) Pub. No.: US 2012/0045807 A1
(43) Pub. Date: Feb. 23, 2012

(54) PROCESS FOR PRODUCING CHEMICALS USING MICROBIAL FERMENTATION OF SUBSTRATES COMPRISING CARBON MONOXIDE

(75) Inventors: Sean D. Simpson, Auckland (NZ); Shane E. Fleming, Auckland (NZ); Alice M Havill, Auckland (NZ); Simon RTrevethick, Auckland (NZ)

(73) Assignee: LANZATECH NEW ZEALAND LIMITED, Auckland (NZ)

(21) Appl. No.: 13/212,891

(22) Filed: Aug. 18, 2011

Related U.S. Application Data
(60) Provisional application No. 61/401,835, filed on Aug. 19, 2010.

(51) Int. Cl. C12P 7/18; C12P 7/26; C12P 7/16; C12P 5/02

(52) U.S. Cl. 435/148; 435/158; 435/167; 435/160

(57) ABSTRACT

A process for converting a substrate such as carbon monoxide to useful chemicals has been developed. The process involves providing a substrate comprising CO to a bioreactor which contains a culture of one or more micro-organisms and anaerobically fermenting the substrate to produce 2,3-butanediol (BDO). The BDO is next converted to one or more of butane, butadiene and/or methyl ethyl ketone which in turn can be converted to other compounds. The source of the CO can be an industrial process such as the ferrous metal products manufacturing. The microorganism can be Clostridium autoethanogenum, Clostridium ljundahlii or Clostridium ragsdalei.

### CROSS REFERENCE TO RELATED APPLICATION

This application claims priority to U.S. Application Ser. No. 61/401,835, filed on Aug. 19, 2010 which is incorporated by reference in its entirety.

### FIELD OF THE INVENTION

The present invention relates to the production of one or more chemical products utilising a step involving microbial fermentation, particularly microbial fermentation of substrates comprising CO.

### BACKGROUND OF THE INVENTION

Butenes are valuable products which are used in the production of a variety of chemicals including fuels and polymers. Butadiene is a valuable resource to produce synthetic rubbers, nylon, and in the synthesis of cycloalkanes and cycloalkenes. Methyl ethyl ketone (or butanone) is a valuable industrial solvent used in the manufacture of plastics, textiles, paraffin wax, lacquers, varnishes, paint removers, and glues, and can be used as a cleaning agent.

Carbon Monoxide (CO) is a major by-product of the incomplete combustion of organic materials such as coal or oil and oil derived products. Although the complete combustion of carbon containing precursors yields CO2 and water as the only end products, some industrial processes need elevated temperatures favouring the build up of carbon monoxide over CO2. One example is the steel industry, where high temperatures are needed to generate desired steel qualities. For example, the steel industry in Australia is reported to produce and release into the atmosphere over 500,000 tonnes of CO annually.

Furthermore, CO is also a major component of syngas, where varying amounts of CO and H2 are generated by gasification of a carbon-containing fuel. For example, syngas may be produced by cracking the organic biomass of waste woods and timber to generate precursors for the production of fuels and more complex chemicals.

The release of CO into the atmosphere may have significant environmental impact. In addition, emissions taxes may be required to be paid, increasing costs to industrial plants. Since CO is a reactive energy rich molecule, it can be used as a precursor compound for the production of a variety of chemicals.

### SUMMARY OF THE INVENTION

It is an object of the present invention to provide a process for the production of one or more chemical products, including processes which produce butene, butadiene, and/or methyl ethyl ketone, by fermenting a substrate comprising CO to produce 2,3-butanediol which is then converted to one or more of the compounds above.

In one embodiment, the method comprises at least:

a. anaerobically fermenting a substrate comprising CO to produce 2,3-butanediol; and,
b. converting the 2,3-butanediol to one or more of butene, butadiene, and/or methyl ethyl ketone.

In one embodiment, the method comprises recovering the 2,3-butanediol after step a, before it is converted to one or more chemical products in step (b).

In one embodiment, the method comprises recovering the intermediate compounds butene, butadiene, and/or methyl ethyl ketone during step (b). In another embodiment, 2,3-butanediol is converted to one or more chemical products without recovery of butene, butadiene, and/or methyl ethyl ketone during step b.

In one embodiment, step (a) comprises providing a substrate comprising CO to a bioreactor containing a culture of one or more micro-organisms and anaerobically fermenting the substrate to produce 2,3-butanediol.

In one embodiment, the method further comprises converting or using butene, butadiene, and/or methyl ethyl ketone in the production of one or more chemical products following recovery of butene, butadiene, and/or methyl ethyl ketone.

In another embodiment, 2,3-butanediol is converted to one or more chemical products without recovery of butene, butadiene, and/or methyl ethyl ketone from the method.

In particular embodiments of the various aspects, the substrate comprising carbon monoxide is a gaseous substrate comprising carbon monoxide. The gaseous substrate comprising carbon monoxide can be obtained as a by-product of an industrial process. In certain embodiments, the industrial process is selected from the group consisting of ferrous metal products manufacturing, non-ferrous products manufacturing, petroleum refining processes, gasification of biomass, gasification of coal, electric power production, carbon black production, ammonia production, methanol production and coke manufacturing. In one embodiment the gaseous substrate comprises a gas obtained from a steel mill. In another embodiment the gaseous substrate comprises automobile exhaust fumes.

In particular embodiments, the CO-containing substrate typically contains a major proportion of CO, such as at least about 20% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present.

In certain embodiments of the various aspects, the method comprises microbial fermentation using a microorganism of the genus Clostridia.

In one embodiment, the method comprises microbial fermentation using Clostridium autoethanogenum.

In one embodiment, the method comprises microbial fermentation using Clostridium ljundahlii.

In one embodiment, the method comprises microbial fermentation using Clostridium ragsdalei.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

These and other objects and embodiments of the invention will become more apparent after the detailed description of the invention.

### BRIEF DESCRIPTION OF THE DRAWINGS

FIG. 1 shows graphs of 2,3-butanediol production for DSM19630 (FIG. 1A) and DSM23693 (FIG. 1B)

FIG. 2 shows graphs of 2,3-butanediol production versus time for C. autoethanogenum, C ljungdahlii and C. ragsdalei.

FIG. 3 shows graphs from the continuous production of products for C. autoethanogenum (DSM23693) from example 3.

### DETAILED DESCRIPTION OF THE INVENTION

The following is a description of the present invention, including preferred embodiments thereof, given in general terms. The invention is further exemplified in the disclosure given under the heading “Examples” herein below, which provides experimental data supporting the invention, specific examples of aspects of the invention, and means of performing the invention.

The term “2,3-butanediol” should be interpreted to include all enantiomeric and diastereomeric forms of the compound, including (R,R), (S,S) and meso forms, in racemic, partially stereoisomerically pure and/or substantially stereoisomerically pure forms.

“Butene” (also known as butylene) as used herein, is intended to refer to all structural isomers of the alkene including 2-butene, but-1-ene, 2-methylpropene, and all stereoisomeric and geometric isomeric forms of the compound, including Z-but-2-ene, E-but-2-ene, in mixtures of isomers and pure and/or substantially pure forms.

As used herein, “butadiene” is intended to refer to all to all geometric isomers of the diene including cis and trans 1,3-butadiene, in mixtures of isomers and pure and/or substantially pure forms.

As used herein, “methyl ethyl ketone” (or MEK or butanone) is intended to refer to all isomers of the ketone in partially pure and/or substantially pure forms.

The phrase “one or more chemical products” is used herein to refer to chemical compounds or products which can be manufactured from or using one or more of butene, butadiene and MEK, and includes products in which one or more of butene, butadiene and MEK are considered intermediates in the production of said products. Various non-limiting examples of such chemical products are provided herein after.

The term “bioreactor” includes a fermentation device consisting of one or more vessels and/or towers or piping arrangement, which includes the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. As is described herein after, in some embodiments the bioreactor may comprise a first growth reactor and a second fermentation reactor. As such, when referring to the addition of a substrate, for example a substrate comprising carbon monoxide, to the bioreactor or fermentation reaction it should be understood to include addition to either or both of these reactors where appropriate.

The term “substrate comprising carbon monoxide” and like terms should be understood to include any substrate in which carbon monoxide is available to one or more strains of bacteria for growth and/or fermentation, for example;

“Gaseous substrates comprising carbon monoxide” include any gas which contains a level of carbon monoxide. The gaseous substrate will typically contain a major proportion of CO, preferably at least about 15% to about 95% CO by volume.

Unless the context requires otherwise, the phrases “fermenting”, “fermentation process” or “fermentation reaction” and the like, as used herein, are intended to encompass both the growth phase and product biosynthesis phase of the process.

In one aspect, the invention provides a method of producing one or more chemical products the method comprising at least the step of anaerobically fermenting a substrate comprising CO to produce 2,3-butanediol. In one embodiment, the method comprises at least anaerobically fermenting a substrate comprising CO to produce 2,3-butanediol and converting the 2,3-butanediol to one or more chemical products via the intermediate compounds butene, butadiene, and/or methyl ethyl ketone.

In another aspect, the invention provides a method of producing one or more of butene, butadiene, and/or methyl ethyl ketone, the method comprising as least anaerobically fermenting a substrate comprising CO to produce 2,3-butanediol. In one embodiment, the method comprises at least anaerobically fermenting a substrate comprising CO to produce 2,3-butanediol and then converting the 2,3-butanediol to one or more of butene, butadiene, and/or methyl ethyl ketone.

In one embodiment, the methods of the invention comprise recovering the 2,3-butanediol from the fermentation broth before it is converted to one or more of butene, butadiene, and/or methyl ethyl ketone. However, in some embodiments, this may not be necessary.

In one embodiment, the methods comprise recovering one or more of butene, butadiene, and/or methyl ethyl ketone produced and following recovery converting or using them in the production of one or more chemical products. In other embodiments, it is not necessary to recover butene, butadiene, and/or methyl ethyl ketone before they are converted or used to produce one or more chemical products.

In one embodiment, the microbial fermentation comprises providing a substrate comprising CO and in a bioreactor containing a culture of one or more micro-organisms, anaerobically fermenting the substrate to produce 2,3-butanediol.

In certain embodiments, the methods of the invention are continuous. In one embodiment 2,3 butanediol is continuously recovered from the fermentation broth or bioreactor. In certain embodiments, the 2,3-butanediol recovered from the fermentation broth or bioreactor is fed directly for chemical conversion to one or more of butene, butadiene and methyl ethyl ketone. For example, the 2,3-butanediol may be fed directly to one or more vessel suitable for chemical synthesis of one or more of butene, butadiene and methyl ethyl ketone. Similarly, in certain embodiments of the invention butene, butadiene, and/or methyl ethyl ketone may be continuously recovered from the method and optionally fed directly to a chemical synthesis reaction for the production of another chemical product. In other embodiments, butene, butadiene, and/or methyl ethyl ketone are converted or used in the production of other chemical products in situ on a continuous basis.

### Microorganisms

Any one or more microorganisms capable of fermenting a substrate comprising CO to produce 2,3 butanediol may be used in the present invention. In one embodiment, the microorganism is of the genus Clostridia.

In certain embodiments of the invention the one or more micro-organisms used in the fermentation is Clostridium autoethanogenum. In certain embodiments the Clostridium autoethanogenum is a Clostridium autoethanogenum having the identifying characteristics of the strain deposited at the German Resource Centre for Biological Material (DSMZ) under the identifying deposit number DMS19630 or the strain deposited at the DSMZ under the identifying deposit number DMS23693. In another embodiment the Clostridium autoethanogenum is a Clostridium autoethanogenum DMS10061 or DMS23693.

In other embodiments, the one or more micro-organism used in the fermentation is Clostridium ljungdahlii or Clostridium ragsdalei. In certain embodiments the Clostridium ljungdahlii has the identifying characteristics of the strain deposited at the German Resource Centre for Biological Material (DSMZ) under the identifying deposit number DMS13582 and the Clostridium ragsdalei has the identifying characteristics of the strain deposited at the American Type Culture Collection (ATCC) under the identifying deposit number ATCC-BAA 622™, however it should be appreciated that other strains may be used.

Culturing of the bacteria used in the method of the invention may be conducted using any number of processes known in the art for culturing and fermenting substrates using anaerobic bacteria. Exemplary techniques are provided in the “Examples” section of this document. By way of further example, those processes generally described in the following articles using gaseous substrates for fermentation may be utilised: K. T. Klasson, M. D. Ackerson, E. C. Clausen and J. L. Gaddy (1991). Bioreactors for synthesis gas fermentations resources. Conservation and Recycling, 5; 145-165; K. T. Klasson, M. D. Ackerson, E. C. Clausen and J. L. Gaddy (1991). Bioreactor design for synthesis gas fermentations. Fuel. 70. 605-614; K. T. Klasson, M. D. Ackerson, E. C. Clausen and J. L. Gaddy (1992). Bioconversion of synthesis gas into liquid or gaseous fuels. Enzyme and Microbial Technology. 14; 602-608; J. L. Vega, G. M. Antorrena, E. C. Clausen and J. L. Gaddy (1989). Study of Gaseous Substrate Fermentation: Carbon Monoxide Conversion to Acetate. 2. Continuous Culture. Biotech. Bioeng. 34. 6. 785-793; J. L. Vega, E. C. Clausen and J. L. Gaddy (1989). Study of gaseous substrate fermentations: Carbon monoxide conversion to acetate. 1. Batch culture. Biotechnology and Bioengineering. 34. 6. 774-784; and, J. L. Vega, E. C. Clausen and J. L. Gaddy (1990). Design of Bioreactors for Coal Synthesis Gas Fermentations. Resources, Conservation and Recycling. 3.149-160.

### Substrates

A substrate comprising carbon monoxide, preferably a gaseous substrate comprising carbon monoxide, is used in the fermentation reaction to produce 2,3 butanediol in the methods of the invention. The gaseous substrate may be a waste gas obtained as a by-product of an industrial process, or from some other source such as from combustion engine (for example automobile) exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing gas may be captured from the industrial process before it is emitted into the atmosphere, using any convenient method. Depending on the composition of the gaseous substrate comprising carbon monoxide, it may also be desirable to treat it to remove any undesired impurities, such as dust particles before introducing it to the fermentation. For example, the gaseous substrate may be filtered or scrubbed using known methods.

In other embodiments of the invention, the gaseous substrate comprising carbon monoxide may be sourced from the gasification of biomass. The process of gasification involves partial combustion of biomass in a restricted supply of air or oxygen. The resultant gas typically comprises mainly CO and H₂, with minimal volumes of CO₂, methane, ethylene and ethane. For example, biomass by-products obtained during the extraction and processing of foodstuffs such as sugar from sugarcane, or starch from maize or grains, or non-food biomass waste generated by the forestry industry may be gasified to produce a CO-containing gas suitable for use in the present invention.

The CO-containing substrate will typically contain a major proportion of CO, such as at least about 15% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present.

It is not necessary for the gaseous substrate to contain any hydrogen, however this is not considered detrimental to 2,3 butanediol production. The gaseous substrate may also contain some CO₂ for example, such as about 1% to about 80% by volume, or 1% to about 30% by volume. In one embodiment it contains about 5% to about 10% by volume. In another embodiment the gaseous substrate contains approximately 20% CO₂ by volume.

Typically, the carbon monoxide will be added to the fermentation reaction in a gaseous state. However, the invention should not be considered to be limited to addition of the substrate in this state. For example, the carbon monoxide could be provided in a liquid. For example, a liquid may be saturated with a carbon monoxide containing gas and then that liquid added to a bioreactor. This may be achieved using standard methodology. By way of example, a microbubble dispersion generator (Hensirisak et. al. Scale-up of microbubble dispersion generator for aerobic fermentation; Applied Biochemistry and Biotechnology Volume 101, Number 3/October, 2002) could be used.

In one embodiment of the invention, a combination of two or more different substrates may be used in the fermentation reaction. In addition, it is often desirable to increase the CO concentration of a substrate stream (or CO partial pressure in a gaseous substrate) and thus increase the efficiency of fermentation reactions where CO is a substrate. Increasing CO partial pressure in a gaseous substrate increases CO mass transfer into a fermentation media. The composition of gas streams used to feed a fermentation reaction can have a significant impact on the efficiency and/or costs of that reaction. For example, O2 may reduce the efficiency of an anaerobic fermentation process. Processing of unwanted or unnecessary gases in stages of a fermentation process before or after fermentation can increase the burden on such stages (e.g. where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

### Media

It will be appreciated that for growth of the one or more microorganisms and substrate to 2,3 butanediol fermentation to occur, in addition to the substrate, a suitable nutrient medium will need to be fed to the bioreactor. A nutrient medium will contain components, such as vitamins and minerals, sufficient to permit growth of the micro-organism used. By way of example only, anaerobic media suitable for the growth of Clostridium autoethanogenum are known in the art, as described for example by Abrini et al (Clostridium autoethanogenum, sp. Nov., An Anaerobic Bacterium That Produces Ethanol From Carbon Monoxide; Arch. Microbiol., 161: 345-351 (1994)). The “Examples” section herein after provides further examples of suitable media.

### Fermentation Conditions

The fermentation should desirably be carried out under appropriate conditions for the substrate to 2,3 butanediol fermentation to occur. Reaction conditions that should be considered include temperature, media flow rate, pH, media redox potential, agitation rate (if using a continuous stirred tank reactor), inoculum level, maximum substrate concentrations and rates of introduction of the substrate to the bioreactor to ensure that substrate level does not become limiting, and maximum product concentrations to avoid product inhibition.

The optimum reaction conditions will depend partly on the particular microorganism of used. However, in general, it is preferred that the fermentation be performed at a pressure higher than ambient pressure. Operating at increased pressures allows a significant increase in the rate of CO transfer from the gas phase to the liquid phase where it can be taken up by the micro-organism as a carbon source for the production of 2,3 butanediol. This in turn means that the retention time (defined as the liquid volume in the bioreactor divided by the input gas flow rate) can be reduced when bioreactors are maintained at elevated pressure rather than atmospheric pressure.

Also, since a given CO-to-product conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

The benefits of conducting a gas-to-product fermentation at elevated pressures have also been described elsewhere. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that the 2,3 butanediol product is consumed by the culture.

Examples of fermentation conditions suitable for anaerobic fermentation of a substrate comprising CO are detailed in WO2007/117157, WO2008/115080, WO2009/022925 and WO2009/064200. It is recognised the fermentation conditions reported therein can be readily modified in accordance with the methods of the instant invention. The inventors have determined that, in one embodiment where pH is not controlled, there does not appear to be a deleterious effect on 2,3-butanediol production.

In one particular embodiment the methodology and conditions described in WO2009/151342 may be used in the present invention.

### Bioreactor

Fermentation reactions may be carried out in any suitable bioreactor as described previously herein. In some embodiments of the invention, the bioreactor may comprise a first, growth reactor in which the micro-organisms are cultured, and a second, fermentation reactor, to which broth from the growth reactor is fed and in which most of the fermentation product (2,3-butanediol, for example) is produced.

### Product Recovery

The fermentation will result in a fermentation broth comprising a desirable product (2,3 butanediol) and/or one or more by-products (such as ethanol, acetate and butyrate) as well as bacterial cells, in a nutrient medium.

In certain embodiments of the reaction, the concentration of 2,3 Butanediol in the fermentation broth is at least 2 g/L, or at least 5 g/L, or at least 10 g/L, or at least 20 g/L.

In certain embodiments the 2,3 butanediol produced in the fermentation reaction is converted to MEK, butene, and/or butadiene directly from the fermentation broth. In other embodiments, the 2,3 butanediol is first recovered from the fermentation broth before conversion to MEK, butene, and/or butadiene.

In certain embodiments, the recovery of 2,3 butanediol comprises continuously removing a portion of broth and recovering 2,3-butanediol from the removed portion of the broth.

In particular embodiments the recovery of 2,3 butanediol includes passing the removed portion of the broth containing 2,3 butanediol through a separation unit to separate bacterial cells from the broth, to produce a cell-free 2,3 butanediol-containing permeate, and returning the bacterial cells to the bioreactor. The cell-free 2,3 butanediol-containing permeate may then be used for subsequent conversion to butene, MEK and/or butadiene.

In certain embodiments, the recovering of 2,3 butanediol and/or one or more other products or by-products produced in the fermentation reaction comprises continuously removing a portion of the broth and recovering separately 2,3 butanediol and one or more other products from the removed portion of the broth.

In some embodiments the recovery of 2,3 butanediol and/or one or more other products includes passing the removed portion of the broth containing 2,3 butanediol and/or one or more other products through a separation unit to separate bacterial cells from the 2,3 butanediol and/or one or more other products, to produce a cell-free 2,3 butanediol- and one or more other product-containing permeate, and returning the bacterial cells to the bioreactor.

In the above embodiments, the recovery of 2,3 butanediol and one or more other products preferably includes first removing 2,3 butanediol from the cell-free permeate followed by removing the one or more other products from the cell-free permeate. Preferably the cell-free permeate is then returned to the bioreactor.

2,3-butanediol, or a mixed product stream containing 2,3 butanediol, may be recovered from the fermentation broth by methods known in the art. By way of example, fractional distillation or evaporation, pervaporation, and extractive fermentation may be used. Further examples include: recovery using steam from whole fermentation broths (Wheat et al. 1948); reverse osmosis combined with distillation (Sridhar 1989); Liquid-liquid extraction techniques involving solvent extraction of 2,3-BD (Othmer et al. 1945; Tsao 1978; Eiteman and Gainer 1989); aqueous two-phase extraction of 2,3-BD in PEG/dextran system (Ghosh and Swaminathan 2003; solvent extraction using alcohols or esters, e.g., ethyl acetate, tributylphosphate, diethyl ether, n-butanol, dodecanol, oleyl alcohol, and an ethanol/phosphate system (Bo Jianga 2009); aqueous two-phase systems composed of hydrophilic solvents and inorganic salts (Zhigang et. al. 2010).

In some cases prior to exposure to solvent, the fermentation broth is dewatered by evaporation (Othmer et al. 1945) or both microfiltration and reverse osmosis (Sridhar 1989) because of the low partition coefficient and the low selectivity of 2,3-butanediol. Repulsive extraction or salting out using potassium chloride (KCl) or dehydrated K2CO3 has also been investigated on the recovery of 2,3-BD (Syu 2001) like the salting-out effect of K2CO3 on extraction of butanol in acetone-butanol-ethanol fermentation (Xu 2001; Hu et al. 2003). The removal of water from the fermentation broth was also tested before salting out because the concentration of 2,3-butanediol in the broth was too low to be salted out even if at a saturated KCl or K2CO3 solution.

A yet further example of a method to recover 2,3-butanediol is to react it with formaldehyde to form a formal under catalysis of acid. The 2,3-butanediol formal is collected in the top oil phase and allowed to react with acid methanol to form 2,3-butanediol and methylal. Methylal can be hydrolyzed to methanol and formaldehyde (Senkus 1946).

A further example, may be the use of ionic liquids to extract the ethanol/2,3-BD from clarified broth. Ionic liquids can be tailored in many ways to change physical properties. An advantage of this approach is that ionic liquids are not volatile. Some are water sensitive but others are not.

Pervaporation or vacuum membrane distillation, used previously in ethanol and butanol fermentations, can be used to concentrate 2,3-BD (Qureshi et al. 1994) in water as an extract from the fermentation broth. A microporous polytetrafluoroethylene (PTFE) membrane is used in the integrated process, while a silicone membrane is usually used in pervaporative ethanol or butanol fermentations.

By-products such as acids including acetate and butyrate may also be recovered from the fermentation broth using methods known in the art. For example, an adsorption system involving an activated charcoal filter or electrodialysis may be used.

In certain embodiments of the invention, 2,3 butanediol and by-products are recovered from the fermentation broth by continuously removing a portion of the broth from the bioreactor, separating microbial cells from the broth (conveniently by filtration, for example), and recovering 2,3 butanediol and optionally other alcohols and acids from the broth. Alcohols may conveniently be recovered for example by distillation, and acids may be recovered for example by adsorption on activated charcoal. The separated microbial cells are preferably returned to the fermentation bioreactor. The cell free permeate remaining after the alcohol(s) and acid(s) have been removed is also preferably returned to the fermentation bioreactor. Additional nutrients (such as B vitamins) may be added to the cell free permeate to replenish the nutrient medium before it is returned to the bioreactor.

Also, if the pH of the broth was adjusted during recovery of 2,3 butanediol and/or by-products, the pH should be re-adjusted to a similar pH to that of the broth in the fermentation bioreactor, before being returned to the bioreactor.

In certain embodiments, the 2,3-butanediol is continuously recovered from the fermentation broth or bioreactor and fed directly for chemical conversion to one or more of butene, butadiene and methyl ethyl ketone. For example, the 2,3-butanediol may be fed directly through a conduit to one or more vessel suitable for chemical synthesis of one or more of butene, butadiene and methyl ethyl ketone or other down stream chemical products.

### Conversion to Chemical Products

A number of known methods may be used for the production of MEK from 2,3 butanediol. For example, MEK can be obtained by the direct dehydration of 2,3-butanediol over a variety of catalysts (sulphuric acid, Cu, AlO3, Zeolite etc): for an example see Emerson et. al. (1982

A number of known methods may be used for the production of butene from 2,3 butanediol. For example, treatment of the diol with HBr, followed by Zn powder results in but-2-ene. The debrominations proceed with a high degree of anti stereospecificity (House and Ro, 1958; Gordon and Hay, 1968), the meso isomer giving the trans butene, and the (+) isomer the cis butene.

A number of known methods may be used for the production of butadiene from 2,3 butanediol. For example, butenes can be catalytically dehydrogenated to 1,3-butadiene in the presence of superheated steam as a diluent and a heating medium (Kearby, 1955). By way of further example, butadiene can also be obtained by the direct dehydration of 2,3-butanediol over thoria catalyst, although most other dehydration catalysts give methyl ethyl ketone as the main product (Winfield, 1945).

Butadiene, butene, and MEK can subsequently be used in a variety of processes for producing commercially useful products.

For example, butene may be used in the production of gasoline and butadiene. By way of yet further example, butene may be used as a component or precursor in the manufacture of C12 paraffins, such as iso paraffins used as aviation fuels (see U.S. Pat. No. 7,338,541, for example).

MEK dissolves many substances and may be used, for example, as a solvent in processes involving gums, resins, cellulose acetate, and nitrocellulose coatings and in vinyl films. For this reason it finds use, for example, in the manufacture of plastics, textiles, paraffin wax, and in household products such as lacquer, varnishes and paint remover, glues, and as a cleaning agent. It also has use as a denaturing agent for denatured alcohol. By way of further example, it may also be used in dry erase markers as the solvent of the erasable dye. In addition, MEK is the precursor to methyl ethyl ketone peroxide, a catalyst used in some polymerization reactions. Further, MEK can be converted to 2-butanol by contacting the MEK with a catalyst such as ruthenium on carbon.

Butadiene may be used, for example, to produce synthetic rubbers and polymer resins. While polybutadiene itself is a very soft, almost liquid material, polymers prepared from mixtures of butadiene with styrene or acrylonitrile, such as ABS, are both tough and elastic. Styrene-butadiene rubber is the material most commonly used for the production of automobile tires. Butadiene may also be used to make nylon via the intermediate adiponitrile, other synthetic rubber materials such as chloroprene, and the solvent sulfolane. In addition, butadiene may be used in the industrial production of 4-vinylcyclohexene via a dimerization reaction and cyclododecatriene via a trimerization reaction. Butadiene is also useful in the synthesis of cycloalkanes and cycloalkenes, as it reacts with double and triple carbon-carbon bonds through the Diels-Alder reaction. By way of further example, butadiene may be used in the manufacture of cycloalkanes, cycloalkenes, dodecandioic acid (DDDA), Adiponitrile, Caprolactam, styrene, ethylidene norbornene, lauryl lactam and 1,5-cyclooctadiene (COD).

It should be appreciated that the methods of the invention may be integrated or linked with one or more methods for the production of downstream products from butene, butadiene and/or MEK. For example, the methods of the invention may feed butene, butadiene and/or MEK directly or indirectly to chemical processes or reactions sufficient for the conversion or production of other useful chemical products. In some embodiments, as noted herein before, 2,3 butanediol is converted to one or more chemical products directly via the intermediate compounds butene, butadiene and/or MEK without the need for recovery of butene, butadiene and/or MEK from the method before subsequent use in production of the one or more chemical products.

In particular embodiments, 2,3-butanediol is converted to butene, butadiene and/or MEK by one or more chemical processes, which in turn is converted to one or more chemical products by one or more chemical processes. In particular embodiments, the one or more chemical products are produced without recovering the butane, butadiene and/or MEK. In another embodiment, 2,3-butanediol is converted to one or more chemical products in a single chemical process via one or more of the butane, butadiene and/or MEK intermediate compounds.

The invention will now be described in more detail with reference to the following non-limiting examples.

### EXAMPLES

### Example 1

### Materials and Methods

Solution A

| | | | | | |
|---|---|---|---|---|---|
| NH₄Ac | 3.083 | g | KCl | 0.15 | g |
| MgCl₂•6H₂O | 0.4 | g | NaCl | 0.12 | g |
| | | | (optional) | | |
| CaCl₂•2H₂O | 0.294 | g | Distilled Water | Up to 1 | L |

Solution B

| | | | | | |
|---|---|---|---|---|---|
| Biotin | 20.0 | mg | Calcium D-(*)- | 50.0 | mg |
| | | | pantothenate | | |
| Folic acid | 20.0 | mg | Vitamin B12 | 50.0 | mg |
| Pyridoxine•HCl | 10.0 | mg | p-Aminobenzoic | 50.0 | mg |
| | | | acid | | |
| Thiamine•HCl | 50.0 | mg | Thioctic acid | 50.0 | mg |
| Riboflavin | 50.0 | mg | Distilled water | To 1 | Litre |
| Nicotinic acid | 50.0 | mg | | | |

Solution C

| | mmol/L | | |
|---|---|---|---|
| Component | H2O | Component | mmol/L H2O |
| FeCl₃ | 0.1 | Na₂SeO₃ | 0.01 |
| CoCl₂ | 0.05 | Na₂MoO₄ | 0.01 |
| NiCl₂ | 0.05 | ZnCl₂ | 0.01 |

### Preparation of Cr (II) Solution

A 1 L three necked flask was fitted with a gas tight inlet and outlet to allow working under inert gas and subsequent transfer of the desired product into a suitable storage flask. The flask was charged with CrCl₃.6H₂0 (40 g, 0.15 mol), zinc granules [20 mesh] (18.3 g, 0.28 mol), mercury (13.55 g, 1 mL, 0.0676 mol) and 500 mL of distilled water. Following flushing with N₂ for one hour, the mixture was warmed to about 80° C. to initiate the reaction. Following two hours of stirring under a constant N₂ flow, the mixture was cooled to room temperature and continuously stirred for another 48 hours by which time the reaction mixture had turned to a deep blue solution. The solution was transferred into N₂ purged serum bottles and stored in the fridge for future use.

### Bacteria

Two types of Clostridium autoethanogenum were used in the following examples. DSM 19630 and DSM 23693, both deposited at the German Resource Centre for Biological Material (DSMZ).

### Sampling and Analytical Procedures

Media samples were taken from the CSTR reactor at intervals over the course of each fermentation. Each time the media was sampled care was taken to ensure that no gas was allowed to enter into or escape from the reactor.

HPLC: HPLC System Agilent 1100 Series. Mobile Phase: 0.0025N Sulfuric Acid. Flow and pressure: 0.800 mL/min. Column: Alltech 10A; Catalog #9648, 150×6.5 mm, particle size 5 μm. Temperature of column: 60° C. Detector: Refractive Index. Temperature of detector: 45° C.

Method for sample preparation: 400 μL of sample and 50 μL of 0.15M ZnSO₄ and 50 μL of 0.15M Ba(OH)₂ are loaded into an Eppendorf tube. The tubes are centrifuged for 10 min. at 12,000 rpm, 4° C. 200 μL of the supernatant are transferred into an HPLC vial, and 5 μL are injected into the HPLC instrument.

Headspace Analysis: Measurements were carried out on a Varian CP-4900 micro GC with two installed channels. Channel 1 was a 10 m Mol-sieve column running at 70° C., 200 kPa argon and a backflush time of 4.2 s, while channel 2 was a 10 m PPQ column running at 90° C., 150 kPa helium and no backflush. The injector temperature for both channels was 70° C. Runtimes were set to 120 s, but all peaks of interest would usually elute before 100 s.

Cell Density: Cell density was determined by counting bacterial cells in a defined aliquot of fermentation broth. Alternatively, the absorbance of the samples was measured at 600 nm (spectrophotometer) and the dry mass determined via calculation according to published procedures.

### A: Batch Fermentation in CSTR

Approximately 1500 mL of solution A was transferred into a 1.5 L fermenter and sparged with nitrogen. Resazurin (1.5 mL of a 2 g/L solution) and H₃PO₄ (85% solution, 2.25 mL) was added and the pH adjusted to 5.3 using concentrated NH₄OH (aq). Nitrilotriacetic acid (0.3 ml of a 0.15M solution) was added prior to 1.5 ml of solution C. This was followed by NiCl2 (0.75 ml of 0.1M solution) and Na₂WO₃ (1.5 mL of a 0.01M solution). 15 ml of solution B was added and the solution sparged with N2 before switching to CO containing gas (42% CO; 36% N2, 2% H2, 20% CO2) at 70 mL/min. The fermenter was then inoculated with 200 ml of a Clostridium autoethanogenum 19630 culture. The fermenter was maintained at 37° C. and stirred at 300 rpm. During this experiment, Na2S solution (0.2M solution) was added at a rate of approx 0.3 ml/hour. Substrate supply was increased in response to the requirements of the microbial culture.

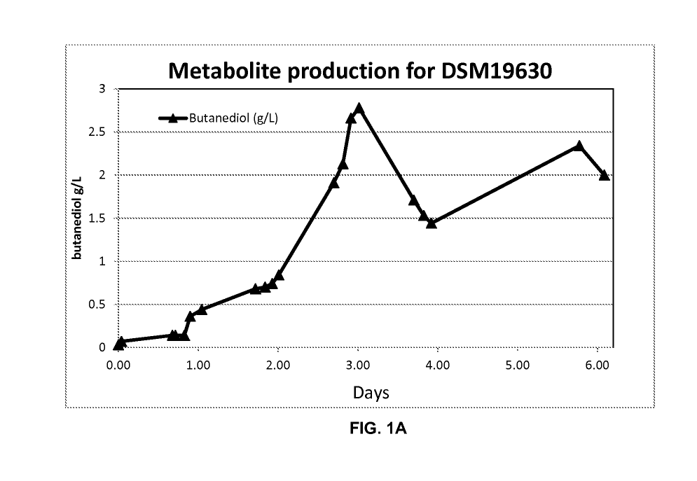

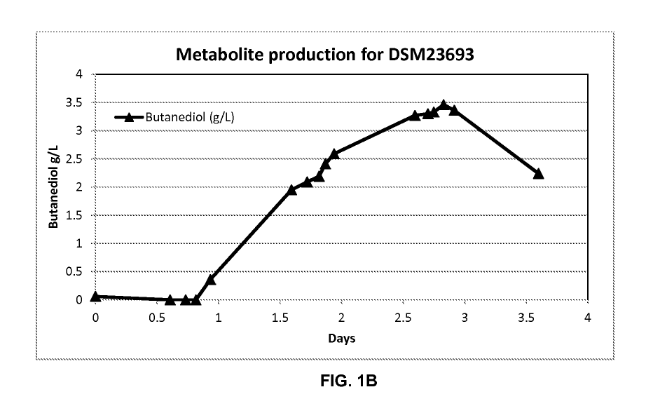

FIG. 1A illustrates 2,3 butanediol was produced by the bacteria.

### B: Batch Fermentation in CSTR

Approximately 1500 mL of solution A was transferred into a 1.5 L fermenter and sparged with nitrogen. Resazurin (1.5 mL of a 2 g/L solution) and H₃PO₄ (85% solution, 2.25 mL) was added and the pH adjusted to 5.3 using concentrated NH₄OH (aq). Nitrilotriacetic acid (0.3 ml of a 0.15M solution) was added prior to 1.5 ml of solution C. Na₂WO₃ (1.5 mL of a 0.01M solution) was added. 15 ml of Solution B was added and the solution sparged with N2 before switching to CO containing gas (42% CO; 58% N2) at 60 mL/min. The fermenter was then inoculated with 180 ml of a Clostridium autoethanogenum 23693 culture. The fermenter was maintained at 37° C. and stirred at 300 rpm. During this experiment, Na2S solution (0.5M solution) was added at a rate of approx 0.12 ml/hour. Substrate supply was increased in response to the requirements of the microbial culture.

FIG. 1B illustrates that 2,3 butanediol was produced by the bacteria.

### Example 2

### Materials and Methods

### Bacterial Strains and Growth Conditions:

C. autoethanogenum DSM 10061 and C. ljungdahlii DSM 13582 were obtained from DSMZ (Deutsche Sammlung von Mikroorganismen and Zellkulturen GmbH) and C. ragsdalei ATCC-BAA 622™ from ATCC (American Type Culture Collection). All organisms were cultivated anaerobically in modified PETC medium (ATCC medium 1754) at 30° C. (C. ragsdalei) or respectively 37° C. (C. autoethanogenum and C. ljungdahlii).

The modified PETC medium contained (per L) 1 g NH4Cl, 0.4 g KCl, 0.2 g MgSO4×7 H2O, 0.8 g NaCl, 0.1 g KH2PO4, 20 mg CaCl2×2 H2O, 10 ml trace elements solution (see below), 10 ml Wolfe's vitamin solution (see below), 2 g NaHCO3, and 1 mg resazurin. After the pH was adjusted to 5.6, the medium was boiled, dispensed anaerobically, and autoclaved at 121° C. for 15 min. Steel mill waste gas (composition: 44% CO, 32% N2, 22% CO2, 2% H2) collected from a New Zealand steel site in Glenbrook, NZ or 0.5% (w/v) fructose (with N2 headspace) were used as carbon source. The media had a final pH of 5.9 and was reduced with Cystein-HCl and Na2S in a concentration of 0.008% (w/v).

The trace elements solution consisted of 2 g nitrilotriacetic acid (adjusted to pH 6 with KOH before addition of the remaining ingredients), 1 g MnSO4, 0.8 g Fe(SO4)2(NH4)2×6 H2O, 0.2 g CoCl2×6 H2O, 0.2 mg ZnSO4×7 H2O, 20 mg CuCl2×2 H2O, 20 mg NiCl2×6 H2O, 20 mg Na2MoO4×2 H2O, 20 mg Na2SeO4, and 20 mg Na2WO4 per liter.

Wolfe's vitamin solution (Wolin et al. 1963) contained (per L) 2 mg biotin, 2 mg folic acid, 10 mg pyridoxine hydrochloride, 5 mg thiamine-HCl, 5 mg riboflavin, 5 mg nicotinic acid, 5 mg calcium D-(+)-pantothenate, 0.1 mg vitamin B12, 5 mg p-aminobenzoic acid, and 5 mg thioctic acid.

### Batch Fermentation in Serum Bottles

Growth experiments were carried out in a volume of 100 ml PETC media in plastic-coated 500-ml-Schott Duran® GL45 bottles with butyl rubber stoppers and 200 kPa steel mill waste gas as sole energy and carbon source. Growth was monitored by measuring the optical density at 600 nm (OD600 nm) and metabolic end products were analyzed by HPLC.

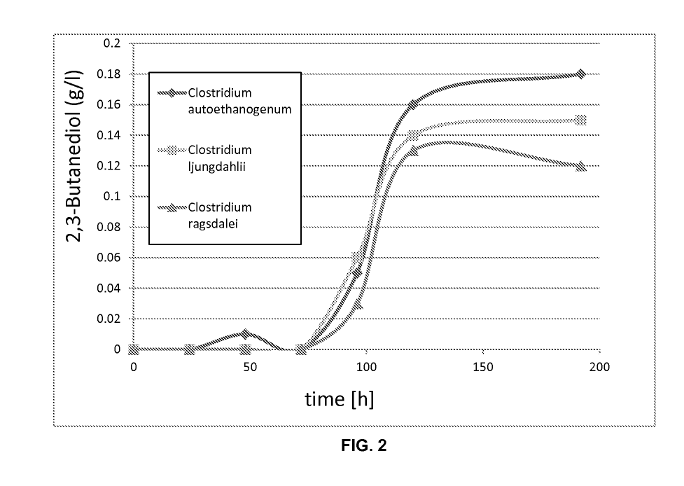

FIG. 2 illustrates that 2,3 butanediol was produced by the various bacteria described above.

### Example 3

### Materials and Methods

### Bacteria:

C. autoethanogenum as deposited at the DSMZ (Deutsche Sammlung von Mikroorganismen and Zellkulturen GmbH) under the accession number DSM23693.

### Sampling and Analytical Procedures:

Media samples were taken from the CSTR reactor at intervals over the course of each fermentation. Each time the media was sampled care was taken to ensure that no gas was allowed to enter into or escape from the reactor.

HPLC: HPLC System Agilent 1100 Series. Mobile Phase: 0.0025N Sulfuric Acid. Flow and pressure: 0.800 mL/min. Column: Alltech 10A; Catalog #9648, 150×6.5 mm, particle size 5 μm. Temperature of column: 60° C. Detector: Refractive Index. Temperature of detector: 45° C.

Method for sample preparation: 400 μL of sample and 50 μL of 0.15M ZnSO₄ and 50 μL of 0.15M Ba(OH)₂ are loaded into an Eppendorf tube. The tubes are centrifuged for 10 min. at 12,000 rpm, 4° C. 200 μL of the supernatant are transferred into an HPLC vial, and 5 μL are injected into the HPLC instrument.

Headspace Analysis: Measurements were carried out on a Varian CP-4900 micro GC with two installed channels. Channel 1 was a 10 m Mol-sieve column running at 70° C., 200 kPa argon and a backflush time of 4.2 s, while channel 2 was a 10 m PPQ column running at 90° C., 150 kPa helium and no backflush. The injector temperature for both channels was 70° C. Runtimes were set to 120 s, but all peaks of interest would usually elute before 100 s.

Cell Density: Cell density was determined by counting bacterial cells in a defined aliquot of fermentation broth. Alternatively, the absorbance of the samples was measured at 600 nm (spectrophotometer) and the dry mass determined via calculation according to published procedures.

### Continuous Fermentation in 500 L Pilot Plant External Loop Reactor

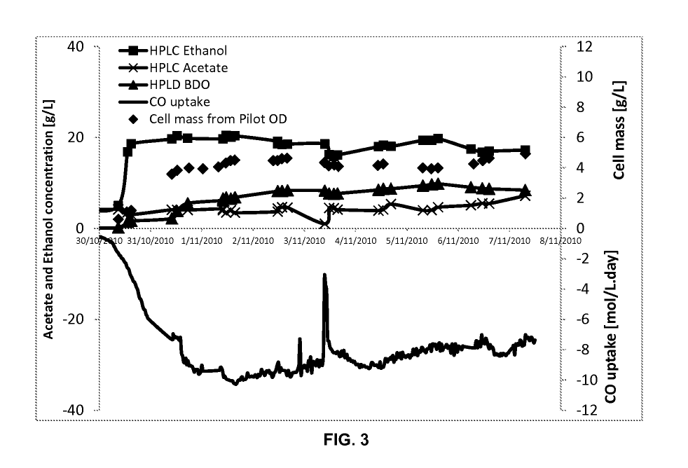

Approximately 500 L of Solution C was added in addition to 1.4 L of Solution A, and 7 L of Solution B, and degassed with N₂ overnight. The fermenter was then fed with a (42% CO, 36% N₂, 2% H₂, 20% CO₂) gas. Fermenter was inoculated with 80 L of a Clostridium autoethanogenum DSM23693 culture. The fermenter was maintained at 37 C. During this experiment, Na₂S solution (0.2 M) was added at a rate of appox. 1 mL/min per kg/hr of gas flow. Gas flow, followed by pump speed and pressure was increased in response to the requirements of the microbial culture. Once the culture reached 2 g/L cell density, it was turned continuous feeding media at 50 L/hr at a dilution rate of 1.8 (day-1) and a cell recycle was started giving a bacterial dilution rate of 0.33 day-1. This fermenter demonstrated a 2:1 ethanol to 2,3 butanediol ratio producing 10 g/L 2,3 butanediol, 20 g/l ethanol and 5 g/l biomass. (FIG. 3)

### Recovery of 2,3 Butanediol-ATPE+Distillation

The first stage of concentrating of the 1% 2,3 Butanediol was by sequential ATPE (Aqueous two phase extraction). 3000 L of Ethanol-stripped fermentation solution was split into 6 IBCs (Intermediate Bulk Containers), each containing 500 L (Labelled IBCs 1 to 6). Into each of the 500 L solutions, 212 Kg of Ammonium Sulphate was added and dissolved by recirculation with a large mechanical pump. Once all 6 IBCs had their salt content added and dissolved, 660 L of Isopropyl Alcohol was added into IBC 1, where it was recirculated around for approximately 10 min and left to settle for approximately 30 minutes. After recirculation and settling, the top phase that formed in IBC 1 (a solvent phase containing Isopropyl Alcohol+2,3Butanediol) was pumped out and directed into IBC 2. This solvent phase was recirculated though IBC 2 for 10 minutes and left to settle for 30 minutes. This recirculation, settling and solvent phase transfer was repeated for IBCs 3, 4, 5 and 6. After all 6 IBCs has been exposed to the solvent solution, the resulting solvent solution was pumped into a spare IBC.

The collected solvent solution was then subjected to distillation for removal of the Isopropyl Alcohol (distillate), leaving behind a 100 L raffinate solution of approx. 17.5% 2,3 Butanediol.

To further purify the 2,3 Butanediol solution, the product was again subjected to further ATPE. This time it was performed in a one-pass batch process with approx. 100 L Isopropyl Alcohol and 50 kg Ammonium Sulphate. The final solvent solution was stripped of the Isopropyl Alcohol and 39 L of a 42% 2,3 Butanediol solution resulted.

The second stage of concentrating the 2,3 Butanediol was via dehydration vacuum distillation. The 39 L 2,3 Butanediol solution at 42% was subjected to distillation under full vacuum at a temperature of approx. 80° C. During this process, the water and acetic acid contents of the 2,3 Butanediol solution were removed as overhead distillate. The remaining product of the distillation process (the raffinate) was concentration 2,3 Butanediol with residual fermentation solids.

The final stage of concentration was evaporation of the 2,3 Butanediol. Under full vacuum and at 120° C., the 2,3 Butanediol was evaporated from the fermentation solids, resulting with a clarified 2,3 Butanediol product. This product equated to a 15 L 2,3 Butanediol solution with a concentration of 98%.

HPLC sampling of the clarified 2,3 Butanediol product were as follows:

| | Phosphoric | Lactic | Acetic | | |
|---|---|---|---|---|---|
| Sample | Acid | Acid | Acid | 23BDO | Ethanol |
| 2,3 | 0 | 0 | 0 | 995.97 g/L | 0 |
| BDO | | | | | |

### Conversion of 2,3 Butanediol to Other Chemicals

In a flow reactor 2,3 butanediol (2,3-BDO) was contacted with gamma-alumina at 300° C. It was observed that the 100% of the 2,3-BDO was converted and yielded 30% methyl ethyl ketone (MEK).

In a flow reactor the MEK from the above experiment was contacted with a catalyst composed of 5 wt. % Ru on carbon. All the MEK was converted and gave a yield of 98% 2-butanol.

A portion of the recovered 2,3-BDO from example 3 above was converted to MEK by contacting it with gamma-alumina at 300° C. in a flow reactor to give 100% conversion of the 2,3-BDO and a yield of about 30%. The other products produced included dimers, trimmers, tetramers of MEK.

The invention has been described herein with reference to certain preferred embodiments, in order to enable the reader to practice the invention without undue experimentation. Those skilled in the art will appreciate that the invention is susceptible to variations and modifications other than those specifically described. It is to be understood that the invention includes all such variations and modifications. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

We claim as our invention:

1. A method for producing one or more chemical products, the method comprising;
a. flowing a substrate comprising CO into a bioreactor containing a culture of one or more microorganisms; and
b. anaerobically fermenting the substrate comprising CO to produce 2,3-butanediol.

2. A method for producing one or more chemical products, the method comprising;
a. flowing a substrate comprising CO into a bioreactor containing a culture of one or more microorganisms;
b. anaerobically fermenting the substrate of step (a) to produce 2,3-butanediol; and
c. converting the 2,3-butanediol produced in step (b) to a compound selected from the group consisting of butane, butadiene, methyl ethyl ketone and mixtures thereof.

3. The method of claim 2 wherein the 2,3 butanediol is recovered before it is converted to the compound in step (c).

4. The method of claim 2, further comprising converting the compound of step (c) into one or more chemical products.

5. The method of claim 4 wherein the 2,3 butanediol is converted to one or more chemical products without recovery of butene, butadiene, and or methyl ethyl ketone.

6. The method of claim 4 where the methyl ethyl ketone is converted to 2-butanol.

7. The method of claim 1 wherein the CO containing substrate comprises at least about 20% CO by volume, to at least about 95% CO by volume.

8. The method of claim 1 wherein the CO containing substrate comprises at least 40% CO.

9. The method according to claim 1 wherein the concentration of 2,3 butanediol produced by the fermentation is at least 2 g/L.

10. The method according to claim 2 wherein the concentration of 2,3 butanediol produced by the fermentation is at least 2 g/L.

11. The method of claim 2 wherein the concentration of 2,3 butanediol is at least 10 g/L.

12. The method of claim 1 wherein the concentration of 2,3 butanediol is at least 20 g/L.

13. The method of claim 2 wherein the concentration of 2,3 butanediol is at least 20 g/L.

14. The method of claim 1 wherein the microorganism is selected from the group comprising Clostridium autoethanogenum, Clostridium ragsdalei, C. ljungdahlii and mixtures thereof.

15. The method of claim 14 wherein the microorganism has the defining features of Clostridium autoethanogenum strain deposited at the German Resource Centre for Biological Material (DSMZ) under the identifying deposit number DSM 23693.

16. The method according to claim 14 wherein the one or more microorganisms is Clostridium autoethanogenum strain deposited DSMZ under the identifying deposit number DSM 23693.

17. The method of claim 2 wherein the microorganism is selected from the group comprising Clostridium autoethanogenum, Clostridium ragsdalei, C. ljungdahlii and mixtures thereof.
